(19) United States
(12) Patent Application Publication
(10) Pub. No.: US 2005/0250098 A1
(43) Pub. Date: Nov. 10, 2005

(54) Method for gene mapping from genotype and phenotype data

(76) Inventors: Hannu T. T. Toivonen, Helsinki (FI); Paivi Onkamo, Helsinki (FI); Kari Vasko, Helsinki (FI); Vesa Ollikainen, Espoo (FI); Pettari Sevon, Helsinki (FI); Heikki Mannila, Espoo (FI); Juha Kere, Stockholm (SE)

Correspondence Address: YOUNG & THOMPSON, 745 SOUTH 23RD STREET, 2ND FLOOR, ARLINGTON, VA, 22202, US

(21) Appl. No.: 10/510,106

(22) PCT Filed: Apr. 1, 2003
(86) PCT No.: PCT/FI03/00248

§ 371 Date: May 10, 2005

(30) Foreign Application Priority Data
Apr. 4, 2002 (FI) 20020651

(51) Int. Cl. C12Q 1/68; G06F 19/00; G01N 33/48; G01N 33/50

(52) U.S. Cl. 435/6; 702/20

(57) ABSTRACT

A method for gene mapping from genotype and phenotype data utilizes linkage disequilibrium between genetic markers mi, which are polymorphic nucleic acid or protein sequences or strings of single-nucleotide polymorphisms deriving from a chromosomal region. All marker patterns P that satisfy a certain pattern evaluation function e(P) are searched from the data, each marker mi of the data is scored by a marker score and the location of the gene is predicted as a function of the scores s(mi) of all the markers mi in the data.

### FIELD OF THE INVENTION

The present invention relates to a method for gene mapping from genotype and phenotype data, which method utilizes linkage disequilibrium between genetic markers mᵢ, which are polymorphic nucleic acid or protein sequences or strings of single-nucleotide polymorphisms deriving from a chromosomal region.

### BACKGROUND OF THE INVENTION

The use of linkage disequilibrium (LD) in detecting disease genes has recently drawn much attention in genetic epidemiology. LD is evaluated with association analysis, which, when applied to disease-gene mapping, requires the comparison of allele or haplotype frequencies between the affected and the control individuals, under the assumption that a reasonable proportion of disease-associated chromosomes has been derived from a common ancestor. Traditional association analysis methods have long been used to test the involvement of candidate genes in diseases and, in special circumstances, to fine-map disease loci found by linkage methods. The testing has mostly been done using simple two-point measures.

Improved statistical methods to detect LD have been presented lately (Terwilliger 1995; Devlin et al. 1996; Lazzeroni 1998; McPeek and Strahs 1999; Service et al. 1999). The newer methods are based on statistical models of LD around a disease susceptibility (DS) gene. Genomic regions—rather than alleles—that are shared among affected individuals, are searched for. The recombination history from the common ancestor to the present day is taken into account with more or less simplified statistical models. The power of these methods, as well as their ability to localize the correct position of the DS gene, has been shown to be better than that of traditional methods. Some of the models are robust against high levels of etiologic heterogeneity (McPeek and Strahs 1999; Service et al. 1999). However, the methods contain assumptions about the inheritance model of the disease and the structure of the survey population, and the effects of violations of these assumptions in the real data are not known. In addition, they can only consider association of one region at a time. Thus, they are currently best suited for fine mapping rather than complex disease mapping or genome screening. The methods also tend to be computationally heavy.

The present inventors have recently introduced a so-called haplotype pattern mining (HPM) method (Toivonen et al. 2000a and 2000b). In the HPM method, haplotype patterns are ordered by their strength of association with the phenotype, and all haplotype patterns exceeding a given threshold level are used for prediction of disease susceptibility gene location. The advantage of the BPM method is that it is model-free as it does not require any assumptions about the inheritance model of the disease. The haplotype patterns are allowed to contain gaps and therefore the HPM method is quite robust against mutations and to missing and erroneous data. How-ever, the basis of the HPM method is that haplotypes, i.e. separate vectors of alleles of markers, are available. As will be explained below, this requirement causes various problems in gene mapping methods, and thus also in the HPM method.

Zhang et al. (2002) have extended the HPM method to allow simultaneous use of haplotype data of related individuals with quantitative trait from an extended pedigree. This is done by employing the Quantitative Pedigree Disequilibrium Test (QPDT) statistic to measure the strength of association between haplotype and a quantitative trait.

The standard procedure in association-based gene mapping is to 1) ascertain individuals carrying the trait of interest and their family members (at least parents), 2) genotype the individuals, 3) derive the haplotypes computationally using genotypes within families, and finally to 4) find associations in the haplotypes (gene mapping).

Even though the actual association analysis is done on sole case and control haplo-types, obtaining these haplotypes requires the parents of the affected individuals to be genotyped as well: vast majority of haplotyping programs available expect the parental genotypes to exist. This means that the parents first have to be recruited, which is not always straightforward, as they might no longer be alive, or cannot be reached, or refuse from giving blood samples. Genotyping more individuals is laborious and elevates the study expenses: per every case or control, 3 individuals will be genotyped instead of just one, so genotyping is done on 3 times as many persons as there are cases and controls. In case the non-transmitted parental chromosomes could be used as controls, a case and his/her parents contributes one case-control pair, in which case the genotyping effort is 1.5 times higher than the number of cases and controls needed.

As an alternative to these haplotyping approaches, some methods for direct haplo-typing from population-based data have indeed been presented, but the problem with these is that they still produce a lot of mistakes, which is a very bad starting point for any haplotype based association program.

There is no straightforward way to use genotypes as input for a method that is designed for haplotypes. Given a genotype, it is in principle possible to consider all haplotype configurations available from the genotype, and to run a haplotype gene mapping method on the different configurations of chromosomes. In practice, however, this is not feasible for marker maps of reasonable size due to a combinatorial explosion: given a genotype with N heterozygous markers, the number of different possible haplotype configurations is 2^{N-1} (or 1, if N=0). For instance, for N=100 the number of possible haplotype configurations is about 6*10²⁹.

Zhang and Zhao (2002) have studied linkage disequilibrium mapping directly with genotype data. Their approach is model-based and the method is based on the decay of haplotype sharing (DHS) method for haplotype data developed by McPeek and Strahs (1999). The approach of Zhang and Zhao is based on explicitly considering all possible haplotype configurations. Since this is not feasible for marker maps of interesting sizes—as was described above—they apply complex and error-prone techniques to prune the number of haplotype configurations they need to consider. Further, in this method, data consisting of multiallele (microsatellite) loci only—thus, no SNPs (single nucleotide polymorphisms) or any other type of markers—is considered. In short, the method works as follows: it is supposed that there are two alleles in the disease locus: the disease causing allele D and the normal allele d. The basic idea is to treat the chromosomes in affected individuals as if they were a random sample from chromosome population consisting of both chromosomes with the D allele and d allele. Chromosomes in normal individuals are assumed to be a random sample of chromosome population only consisting of d chromosomes. Next, a likelihood for individual haplotypes is formulated in the same way as presented by McPeek and Strahs (1999) where the probability of an observed haplotype is dependent of number of generations from original disease mutation, recombination rates between markers, and mutation rate in marker loci. The gap between using haplotype data as the starting point (as in McPeek and Strahs) and genotype data (as Zhang and Zhao present) as the starting point is bridged with following inference: for each genotype gᵢ, there are several haplotype pairs that are compatible with it (2^{N-1}, where N is the number of heterozygote sites in the genotype). The likelihood for an observed genotype is the sum of probabilities of each possible haplotype pair, where the probabilities of individual haplotypes are formulated as above. The genetic parameters of interest (such as location of the disease locus, mutation rate and disease allele age) are then estimated by using EM algorithm. The large number of possible ancestral haplotypes prerequisites pruning out any too rare haplotypes; the haplotype frequencies are estimated with Markov model, and any which are below some prespecified level are left unconsidered.

The approach of Zhang and Zhao has the following serious drawbacks. First, the principle of Zhang and Zhao is to explicitly consider all possible haplotype configurations. This is feasible only with very small marker maps. Second, to avoid the first problem and to extend the applicability of the approach to larger maps, Zhang and Zhao apply additional pruning techniques to reduce the number of haplotype configurations they need to consider. However, those techniques are complex and error-prone. Third, their approach is based on summing probabilities of different haplo-type configurations. Such an approach is not directly applicable to pattern-based mapping methods such as HPM.

Curtis et al. (2001) studied the use of an artificial neural network to detect association between disease and multiple marker genotypes. The pattern-recognition properties of the network were used in the hope that marker haplotypes implicit in the genotypes differed between cases and controls in a way which led to the network being able to classify the subjects correctly, according to their marker genotype.

### SUMMARY OF THE INVENTION

The object of the present invention is to provide a model-free and computationally effective method allowing direct association analysis on genotype rather than haplo-type data, which overcomes the above-mentioned drawbacks. The invention offers remarkable advantages by avoiding the technically difficult, costly and sometimes impossible steps of recruiting and genotyping family members, as well as by avoiding some of the error sources present in population-based haplotyping methods.

The above-mentioned object is achieved in accordance with the invention by the method for gene mapping from genotype and phenotype data, which utilizes linkage disequilibrium between genetic markers mi, which are polymorphic nucleic acid or protein sequences or strings of single-nucleotide polymorphisms deriving from a chromosomal region. The method according to the invention is characterized by the following steps:
i) all marker patterns P that satisfy a pattern evaluation function e(P) are searched from the data, wherein
   a. the marker patterns are expressions involving the marker-allele assignments and zero or more of the following: individual covariates, environmental variables and auxiliary phenotypes; and
   b. the pattern evaluation function e(P) involves some statistical measure of the association between the marker pattern P and the phenotype being studied,
   by testing each marker of pattern P against the corresponding allele pair in genotype G, effectively finding out if there is a possible haplotype configuration of G which matches P and counting the possible matches as matches,
ii) each marker mᵢ of the data is scored by a marker score s(mᵢ), which is a function of the set Sᵢ defined as the set of marker patterns overlapping the marker mᵢ and satisfying the pattern evaluation function e as defined in step (i), and
iii) the location of the gene is predicted as a function of the scores s(mᵢ) of all the markers mᵢ in the data and is based on maximizing the score if the scoring function is designed to give higher scores closer to the gene, and on minimizing the score if the scoring function is designed to give lower scores closer to the gene, as is the case for instance when the scores s(mᵢ) are marker-wise p values. A computer-readable data storage medium according to the invention has computer-executable program code stored thereon, said executable program code being operative to perform a method of any embodiments of the invention when executed on a computer.

A computer system according to the invention is programmed to perform the method of any embodiments of the invention.

As used herein the term ‘haplotype’ defines a vector of alleles in a single chromosome. Also, as used herein the term ‘genotype’ defines a vector of (unphased) allele pairs in a chromosome pair.

The term ‘microsatellite’ used defines a small run (usually less than 0.1 kb) of tandem repeats of a very simple DNA sequence, usually 1-4 bp, for example (CA)n. It has been used as the primary tool for genetic mapping during the 1990s. ‘Multiallelic genetic locus’ is a gene with high level of variation; there are several types of variants in the gene locus, each with reasonably high frequency. ‘SNP’, single nuleotide polymorphism, defines any polymorphic variation at a single nucleotide. Although less informative than microsatellites, SNPs are more amenable to large-scale automated scoring.

### BRIEF DESCRIPTION OF THE DRAWINGS

FIG. 1 shows the localization accuracy of HPM-G compared to HPM: the y axis shows which fraction of simulated data sets is in the predicted region, the length of which is given on the x axis.

FIG. 2 shows the effect of sample size on localization accuracy with a) HPM-G (sample size in people) and b) HPM (sample size in chromosomes).

FIG. 3 shows the effect of missing data (5%, 10%) on localization accuracy with a) HPM-G (150 affected and 150 control individuals) and b) HPM (200 disease-associated and 200 control chromosomes).

FIG. 4 shows the effect of 100 permutations on localization accuracy.

### DETAILED DESCRIPTION OF THE INVENTION

The object of the present invention is to provide a method for gene mapping from genotype and phenotype data, which utilizes linkage disequilibrium between genetic markers mᵢ, which are polymorphic nucleic acid or protein sequences or strings of single-nucleotide polymorphisms deriving from a chromosomal region. The chromosome data may consist of genotypes or haplotypes. The phenotype being studied may also be a combination of several phenotypes.

The method according to the invention, also named as haplotype pattern mining in genotype data (HPM-G), uses data mining methods in LD-based gene mapping. The method uses both genotypes and haplotypes as input. In diseases with reasonable genetic contribution, affected individuals are likely to have higher frequencies of associated marker alleles near the DS gene than control individuals. Combinations of marker alleles which are more frequent in genotypes of affected individuals than in genotypes of unaffected individuals, are searched for in the data, without assumptions about the mode of inheritance of the disease. These combinations, marker patterns or haplotype patterns, are sorted by the strength of their association with the disease, and the resulting list of marker or haplotype patterns is used in localizing the DS gene. Terms marker pattern and haplotype pattern denote the same concept, and are used interchangeably in this text.

The method according to the present invention is an algorithm-based extension of traditional association analysis. It works with a non-parametric statistical model and without any genetic models. The localization power of the method of the invention is high, even in cases, where multiple independent founder mutations are allowed, and the frequency of the most common mutation in the affected chromosomes varies between 5-15% at realistic sample sizes (100 affected individuals and a similar number of population controls). In addition, the experiments suggest that the method is highly robust against missing data. Since HPM-G can handle high degrees of etiologic heterogeneity, it can be successful in complex disease mapping.

LD, the non-random association of marker alleles and haplotypes to the disease, is likely to be strongest around the DS gene; consequently the locus is likely to be where most of the strongest associations are. In the HPM-G method according to the invention, we search for shared, flexible haplotypes that may contain gaps, and find out, which ones are strongly associated with the disease status. We then use a non-parametric model for predicting the DS locus, on the basis of the locations of the haplotypes. Permutation tests can be used to contrast the results against the null hypothesis that there is no gene effect.

Marker or Haplotype Patterns and Disease Association

We examine linkage disequilibrium by looking for marker or haplotype patterns that consist of a set of nearby markers, not necessarily consecutive ones. Given a marker map M with k markers m₁, . . . , mₖ, a “marker pattern” or “haplotype pattern” P on M is defined as a vector (p₁, . . . , pₖ), where each pᵢ is either an allele of marker mᵢ or the “don't care” symbol (*). The haplotype pattern P occurs in a given haplotype vector (chromosome) H=(h₁, . . . , hₖ) if pᵢ=hᵢ or pᵢ=* for all i,1<=i<=k. Pattern P occurs in a given genotype G=({g₁₁, g₁₂}, . . . , {gₖ₁,gₖ₂}) if pᵢ=gᵢ₁ or pᵢ=gᵢ₂ or pᵢ=* for all i,1<=i<=k.

For example, consider a marker map of 10 markers. The vector P₁=(*, 2, 5, *, 3, *, *, *, *, *), where 1, 2, 3, . . . are marker alleles, is an example of a haplotype pattern. This pattern occurs, for instance, in a chromosome with haplotype (4, 2, 5, 1, 3, 2, 6, 4, 5, 3). The pattern also occurs in the genotype ({2,5}, {2,3}, {1,5}, {4,6}, {3,6}, {2,4}, {1,2}, {1,4}, {3,5}, {1,6}). (For instance, {2,5} is the genotype of marker 1; the alleles are 2 and 5, but their phases are not known.)

Our goal is to search for haplotype patterns that roughly correspond to haplotypes identical by descent in the disease-associated. In doing this, there are two major issues with respect to the shapes of haplotype patterns: the genetic length of the significant part of the patterns, and gaps. We define the “(genetic) length” of a haplo-type pattern P=(p₁, . . . , pₖ) as the maximum distance, in Morgans, between any two markers mᵢ,mⱼ with pᵢ≈*≈pⱼ. Searching for haplotype patterns of arbitrary length hardly makes sense; it is unlikely that genetically extremely long patterns will be discovered, at least not in significant numbers. Consequently, when haplotype patterns are searched for, the maximum length of patterns to be considered can be constrained with an optional pattern-search parameter to the HPM-G method.

We allow for gaps in the marker patterns, since mutations, gene conversions, errors, missing data, and recombinations can corrupt continuous haplotypes. Marker mutations and errors typically cause very short gaps only. Missing information can span several consecutive markers, depending on the data collection scheme. Longer gaps can be introduced by double recombinations which, however, are rare on genetically short distances. In the HPM-G method, the maximum number and maximum length of gaps can be controlled with pattern search parameters.

Mining Disease-Associated Haplotype Patterns

All marker patterns P that satisfy a pattern evaluation function e(P) are searched from the data in the step (i) of the method of the invention. The pattern evaluation function e(P) involves some statistical measure of the association between the marker pattern P and the phenotype being studied. In step (ii), each marker mᵢ of the data is scored by a marker score s(mᵢ), which is a function of the set Sᵢ defined as the set of marker patterns overlapping the marker mᵢ and satisfying the pattern evaluation function e as defined in step (i).

In step (i), let U be the universe of marker patterns considered in the study. The output S of this phase is the set of those marker patterns that satisfy e, i.e., S=(P ε U|e(P) is true}.

In step (ii), for each marker mi in the data, let Sᵢ={P ε U|P makes a reference to mᵢ, or to a marker to the left of and to a marker to the right of mᵢ} be the set of patterns in S that overlap with the marker mᵢ. In this phase each marker mᵢ is scored as a function of Sᵢ, and the result is s(mᵢ).

In step (iii), the location of the gene is predicted as a function of the scores s(mᵢ) of all the markers mᵢ in the data. This function returns an area where the gene is likely to be. The area can be contiguous or fragmented, and it can be a point in a special case.

The marker or haplotype patterns P can be searched for by an algorithm developed by the inventors for this purpose or by the levelwise search method described in Mannila and Toivonen (1997). Preferred algorithms are given in the following.

Version 1 of the Algorithm for Marker Pattern Searching

The following algorithm is a simple, generic, and efficient way to implement step (i) of the method according to the invention. It is based on depth-first search in the space of patterns, a standard procedure in computer science. A pre-requisite is that 10 there is a suitable generalization relation for the patterns, such that if a pattern satisfies the evaluation function, then all more general patterns also satisfy it.

Input
set U of possible marker patterns
evaluation function e(P) for patterns P in U
(generalization) relation<for patterns in U
where the function e and the relation<are such that if e(P) is true and P′<P, then e(P′) is also true

Output
set S={P ε U|e(P) is true} of patterns

Method

1. S : = { }
2. // Initialize the set of evaluated patterns:
3. E := { }
4. // Start with the most general patterns:
5. Gen := {P in U | there is no P′ in U, P′ != P, such that P′ < P}
6. // Recursively evaluate patterns in a depth first order:
7. foreach P ∈ Gen { evaluatePatterns(P) }
8. end;
9. procedure evaluatePatterns(P) {
10. insert P into the set E
11. if e(P) = true then {
12. insert P into set S
13. // Find all specializations of P that have not been tested yet,
14. // and evaluate them recursively:
15. Spec := {P′ in U-E | P < P′, P′ != P, and there is no
16. P″ in U-E, P″ != P and P″ != P′, with P < P″ < P′};
17. foreach P′ in Spec { evaluatePatterns(P′); }
18. }
19. }

Version 2 of the Algorithm for Marker Pattern Searching

The following algorithm is a simple, generic, and efficient way to implement step (i) of the method according to the invention. It is based on depth-first search in the space of patterns, a standard procedure in computer science. It approximates the exact answer by ignoring infrequent and therefore statistically less important patterns.

Define an auxiliary evaluation function ae(P) which is true if and only if the frequency of pattern P exceeds a given threshold x, (how to specify the threshold is discussed elsewhere) and replace the original evaluation function e(P) by function e′(P) defined as e′(P)=true if and only if e(P) is true and ae(P) is true. This refinement prunes patterns that satisfy e but are no more frequent than x. Such infrequent patterns are statistically not relevant, and therefore little information is lost when they are ignored. Now a suitable generalization relation is obtained from logical implication based on the pattern syntax: P<P′ if and only if P′−>P.

The algorithm uses the generalization relation based on logical implication to structure the search space, and the auxiliary function ae to prune the search space. All patterns satisfying ae are searched for, but only those also satisfying e are output.

Input
set U of possible marker patterns
evaluation function e(P) for patterns P in U
frequency threshold x

Output
set S={P in U|e(P) and ae(P) is true} of patterns, where ae(P) is true if and only if the frequency of pattern P exceeds a given threshold x

Method

20.S : = { }
21.// Initialize the set of evaluated patterns:
22.E := { }
23.// Start with the most general patterns:
24.Gen := {P in U | there is no P′ in U, P′ != P, such that P -> P′ }
25.// Recursively evaluate patterns in a depth-first order:
26.foreach P in Gen { evaluatePatterns(P) }
27.end
28.procedure evaluatePatterns(P) {
29. insert P into the set E
30. if ae(P) = true then {
31. if e(P) = true then insert P into set S
32. // Find all specializations of P that have not been tested yet,
33. // and evaluate them recursively:
34. Spec := {P′ in U-E | P′ -> P, P′ != P, and there is no P″ in
35. U-E, P″ != P and P″ != P′, with P′ -> P″ and P″ -> P }
36. foreach P′ in Spec { evaluatePatterns(P′) }
37. }
38. }

Version 3 of the Algorithm for Marker Pattern Searching

When phenotype being studied is qualitative and the pattern evaluation function e(P) has the form e(P)=true if and only if e′(P)>x, where e′(P) is the (signed) association measure χ² and x is a user-specified minimum value, which is chosen so that the sizes of Sᵢ are large enough, such as 7, to give statistically sufficiently reliable estimates for the gene locus, the following algorithm is a simple, generic, and efficient way to implement step (i) of the method according to the invention. It is based on depth-first search in the syntactic space of patterns. It derives a lower bound lb for pattern frequency from the given lower bound x for chi-squared test, and uses lb to prune the search.

Input
marker map M=(m₁, . . . , mₖ)
phenotype vector Y=(Y₁, . . . , Yₙ)
genotype matrix H of size n*k*2 (n persons, k markers, 2 alleles per person and marker)
association threshold x for chi-squared test
maximum pattern length l
maximum number of gaps g
maximum gap size s

Output
set S={P in U|e(P) is true} of patterns,
where U consists of patterns on M that consist of marker-allele assignments and that adhere to parameters l, g, and i, and
where e(P) is true if and only if chi-squared test on P using genotype matrix H and phenotypes Y exceeds the given threshold x

Method

39.S : = { }
40.// Number of case and control persons:
41.pi_{A} := number of affected persons;
42.pi_{C} := number of control persons;
43.pi := pi_{A} + pi_{C}
44.// A lower bound for pattern frequency:
45.lb := pi_{A}* pi * x / (pi_{C} * pi + pi_{A} * x)
46.// Variable for iterating over different patterns:
47.P = (p1, ... , pk) := (‘*’, ... , ‘*’)
48.for i := 1 to k {
49. // alleles(mᵢ) is the set of alleles of the i:th marker
50. foreach a in alleles(mᵢ) {
51. pᵢ := a
52. // Test pattern P and all its extensions:
53. checkPatterns(P, i, i, 0, 0)
54. // Reset pᵢ:
55. pᵢ := ‘*’
56. }
57. }
58.end
59.// Test haplotype pattern P and all patterns that can be generated by
60.// extending P from the right:
61.procedure checkPatterns(P, start, i, nr_of_gaps, gap_length) {
62. // Output strongly associated patterns
63. if chi-squared(P, M, H, Y) >= x and pᵢ != ‘*’ then insert P into set S
64. // Return if extended patterns would be too long:
65. if i = k or i+1-start > l then return
66. // Return if extended patterns can not be strongly disease-associated:
67. if frequency of P in disease-associated persons is less than lb
68. then return;
69. // Create and test legal extensions of current pattern P (3 cases):
70. // 1. Give marker i+1 all possible values:
71. foreach a in alleles(mᵢ₊₁) {
72. pᵢ₊₁ := a
73. checkPatterns (P, start, i+1, nr_of_gaps, 0)
74. }
75. // 2. Introduce a new gap starting at marker i+1:
76. if pᵢ ≠ ‘*’ and nr_of_gaps < g and s ≧ 1 then {
77. pᵢ₊₁ := ‘*’
78. checkPatterns (P, start, i+1, nr_of_gaps+1, 1)
79. }
80. // 3. Extend the current gap over marker i+1:
81. if pᵢ = ‘*’ and gap_length < s then {
82. pᵢ₊₁ := ‘*’
83. checkPatterns (P, start, i+1, nr_of_gaps, gap_length+1)
84. }
85. // Before returning, reset pᵢ₊₁:
86. pᵢ₊₁ := ‘*’
87. return
88. }

Version 4 of the Algorithm for Marker Pattern Searching

The following algorithm is a simple, generic, and efficient way to implement step (i) of the method according to the invention. It is based on the levelwise search method described in Mannila and Toivonen (1997).

Input
set U of possible marker patterns
evaluation function e(P) for patterns P in U
(generalization) relation<for patterns in U, where the function e and the relation<are such that if e(P) is true and P′<P, then e(P′) is also true

Output
set S={P in U|e(P) is true} of patterns

Definitions
function Lgg: U→2^{U}, Lgg(P)={P′ in U|P>P′ and P′ !=P and there is no P″ in U such that P !=P″ !=P′ and P>P″>P′), the set of least general generalizations of pattern P.
function Lss: U→2^{U}, Lss(P)={P′ in U|P<P′ and P′ !=P and there is no P″ in U such that P !=P″!=P′ and P<P″<P′), the set of least special specializations of pattern P.

Method

89.S : = { }
90.Q : = { }
91.// Start with the most general patterns:
92.F : = {P in U | there is no P′ in U, P′ != P, such that P′ < P};
93.while F != { } {
94. // Evaluate the candidate patterns:
95. foreach P in F {
96. if e(P) = true then insert P into set S
97. else remove P from set F
98. }
99. Q : = Q union F
100. // Generate a new set of candidate patterns:
101. C : = { }
102. foreach P in F {
103. C : = C union { P′ in U | P′ in Lss(P) and for all
104. P″ in Lgg(P′): P″ in Q }
105. }
106. F : = C
107. }
108. end

Version 5 of the Algorithm for Marker Pattern Searching

This is the levelwise search version of the algorithm 2.

Input
set U of possible marker patterns
evaluation function e(P) for patterns P in U
frequency threshold x

Output
set S={P in U|e(P) and ae(P) is true} of patterns, where ae(P) is true if and only if the frequency of pattern P exceeds a given threshold x

Definitions
function Lgg: U→2^{U}, Lgg(P)={P′ in U|P→P′ and P′ !=P and there is no P″ in U such that P !=P″ !=P′ and P→P″→P′}, the set of least general generalizations of pattern P.
function Lss: U→2^{U}, Lss(P)={P′ in U|P′→P and P′ !=P and there is no P″ in U such that P !=P″!=P′ and P′→P″→P}, the set of least special specializations of pattern P.

Method

109. S : = { }
110. Q : = { }
111. // Start with the most general patterns:
112. F := {P in U | there is no P′ in U, P′ != P, such that P -> P′ };
113. while F != { } {
114. // Evaluate the candidate patterns:
115. foreach P in F {
116. if ae(P) = true then {
117. if e(P) = true then insert P into set S
118. }
119. else remove P from set F
120. }
121. Q : = Q union F
122. // Generate a new set of candidate patterns:
123. C : = { }
124. foreach P in F {
125. C : = C union { P′ in U | P′ in Lss(P) and for all
126. P″ in Lgg(P′): P″ in Q }
127. }
128. F : = C
129. }
130. end

The phenotype being studied may be qualitative, for example disease is present or disease is absent. In that case, the pattern evaluation function e(P) may have the form e(P)=true if and only if e′(P)>x, where e′(P) is the (signed) association measure χ² and x is a user-specified minimum value, which is chosen so that the sizes of Sᵢ are large enough, such as 7, to give statistically sufficiently reliable estimates for the gene locus and the score s(mᵢ) of marker mᵢ is the size of Sᵢ, also called marker-wise pattern frequency of mᵢ and denoted by f(mᵢ).

As mentioned above, the (signed) χ² is a measure of marker-disease association. A signed version of the measure is used in order to discriminate disease association from control association. The signed χ² measure ±χ²(P) of a haplotype pattern P is positive if P is more frequent in cases than in controls, and negative otherwise. Given a “(positive) association threshold” x, we say that P is “strongly associated” with the disease if ±χ²(P)≧x.

The first part of the HPM-G method can be described as follows. Given the data—markers M, genotypes H, and phenotypes Y—the task is to output all haplotype patterns P that are strongly associated with the disease status for a given value of the association threshold x. We denote the collection of all such haplotype patterns by S—that is, S={P is a haplotype pattern on M|±χ²(P)≧x}. If pattern parameters are specified—a maximum genetic length, a maximum number of gaps, or a maximum length for gaps—the task is refined by requiring that these additional restrictions are also fulfilled.

The signed χ² value is calculated from a 2×2 contingency table, where the rows correspond to the trait-association statuses of the persons, and the columns correspond to the presence and absence of the haplotype pattern. A pattern P=(p₁, . . . , pₖ) is present in a given genotype G=({g₁₁, g₁₂}, . . . , {gₖ₁,gₖ₂)) if pᵢ=gᵢ₁ or pᵢ=gᵢ₂ or pᵢ=* for all i, 1<=i<=k. If, instead of a genotype, two haplotype vectors H₁=(h₁ₗ, . . . , h₁ₖ) and H₂=(h₂ₗ, . . . , h₂ₖ) are given for a person, pattern P is considered to be present in the person if it is present in either of the haplotypes, i.e., if either pᵢ=h₁ᵢ or pᵢ=* for all i,1<=i<=k, or pᵢ=h₂ᵢ or p₁=* for all i,1<=i<=k.

The value of χ² statistic is computed normally, and a negative sign is attached, if the relative frequency of the haplotype pattern among the control persons is higher than among the trait-associated persons.

The first observation in solving the pattern-mining task is that given an association threshold x, a lower bound can be derived for the frequency of strongly associated haplotype patterns as follows:

Given a 2×2 contingency table of the numbers of disease-associated (A) and control (C) persons either matching a pattern (P) or not (N), the χ² test statistic for the disease association of the pattern is defined by

$$\frac{{( {π}_{AP} \cdot {π}_{CN} - {π}_{AN} \cdot {π}_{CP} )}^{2} \cdot π}{{π}_{A} \cdot {π}_{C} \cdot {π}_{P} \cdot {π}_{N}} ,$$

where πᵢⱼ is the number of persons with properties i and j, πᵢ the number of persons with property i, and π the total number of persons. Given the number of affected persons (π_{A}), the number of control persons (π_{C}), and a lower bound x for the test statistic, we can derive a lower bound for the pattern frequency among the affected persons (π_{AP}) as follows. Assuming the pattern is disease-associated, we have π_{AP}·π_{CN}>π_{AN}·π_{CP}. The test statistic is maximized when π_{CP}=0, implying π_{AP}=π_{P} and π_{CN}=π_{C}. Then

$$\frac{{( {π}_{AP} \cdot {π}_{CN} - {π}_{AN} \cdot {π}_{CP} )}^{2} \cdot π}{{π}_{A} \cdot {π}_{C} \cdot {π}_{P} \cdot {π}_{N}} = \frac{{( {π}_{AP} \cdot {π}_{C} )}^{2} \cdot π}{{π}_{A} \cdot {π}_{C} \cdot {π}_{AP} \cdot ( π - {π}_{P} )} = \frac{{π}_{AP} \cdot {π}_{C} \cdot π}{{π}_{A} \cdot ( π - {π}_{AP} )}$$

$$and$$

$$\frac{{π}_{AP} \cdot {π}_{C} \cdot π}{{π}_{A} \cdot ( π - {π}_{AP} )} \geq x ⇒ {π}_{AP} \geq \frac{{π}_{A} \cdot π \cdot x}{{π}_{C} \cdot π + {π}_{A} \cdot x} .$$

The situation is symmetric for protective haplotypes, and the lower bound for π_{CP} is obtained by simply swapping π_{A} and π_{C} in the above result. If disease-associated and protective haplotypes are searched for at the same time, the smaller of π_{AP} and π_{CP} can be used as a lower bound for π_{P}, making the implementation slightly simpler.

On another hand, given such a frequency threshold, all patterns exceeding the threshold can be enumerated efficiently with data-mining algorithms or a standard depth-first search method. An algorithm that first finds all haplotype patterns whose frequency exceeds the computed lower bound and then evaluates the association measure on them, is guaranteed to find the exact set of strongly disease-associated patterns.

The approach is suitable for finding protective haplotype patterns by considering patterns P with ±χ²(P)≦−x. The derivation of the lower bound for the frequency among controls is identical to the case above. Obviously, both disease-associated and protective haplotypes can be found when |±χ²(P)|≧x.

In another embodiment according to the invention, the phenotype being studied may be, in addition to qualitative, also quantitative, for example a measured blood concentration of a substance has a certain value. In that case, the pattern evaluation function e(P) may have the form e(P)=true if and only if e′(P)>x, where e′(P) is the absolute frequency of pattern P in the data and x is a user-specified value, which is chosen so that the sizes of Sᵢ are large enough, such as 20, to give statistically sufficently reliable estimates for the gene locus. In this embodiment, the statistical strength of the method may be still increased.

A linear model is of form Y=β₁X₁+ . . . +βₖXₖ+αZ+β₀, where the dependent variable Y is a quantitative phenotype, X₁ through Xₖ are covariates, such as enviromental factors, and Z is a dummy variable for the occurrence of the haplotype pattern. Firstly, the coefficients α and β* are adjusted for best fit. Secondly, the significance of Z as a covariate is assessed by using a t test. If the phenotype is dichotomous, then the logit transformation can be applied.

Marker Scoring in the Case of Qualitative Phenotype Being Studied

Haplotype patterns close to the DS locus are likely to have stronger association than haplotypes further away; consequently the locus is likely to be where most of the strongest associations are. In one embodiment according to the invention, the marker score s(mᵢ) is defined as the marker frequency f(mᵢ) of marker mᵢ (with respect to M,H,Y,x) as the number of patterns that contain marker mᵢ, possibly mi in a gap: f(mᵢ)=|{P=(p₁, . . . , pₖ)εS | there exist t≦i and u≧i such that pₜ≈*≈pᵤ}|. The idea is that each haplotype pattern roughly corresponds to a continuous chromosomal region, potentially identical by descent, where gaps allow for corruption of marker data. While markers within gaps are not used in measuring the disease association of the pattern, the whole chromosomal region of the pattern is thought to be relevant.

Marker Scoring in the Case of Qualitative or Quantitative Phenotype Being Studied

In order to derive the score s(mᵢ), the p value (statistical significance) of each marker pattern P in determining the phenotype being studied is evaluated, and the score s(mᵢ) is the distance between the observed p value distribution of patterns in Sᵢ and the uniform distribution, defined as average of (pᵢ−qᵢ) log (pᵢ/qᵢ) over all i=1 . . . n, where n is the number of haplotype patterns in Sᵢ, pᵢ is the ith smallest p value in Sᵢ, and qᵢ is the expectation of the ith smallest p value, if the p values were randomly drawn from the uniform distribution.

Gene Localization

The location of the gene, predicted as a function of the scores s(mᵢ) and based on maximizing or minimizing the score, is predicted to
the location of the marker mᵢ that maximizes or minimizes the marker score s(mᵢ), or
the combination of most probable intervals for containing the trait-susceptibility locus that covers at most the desired proportion t (tε {0,100%}) of the original region obtained by taking all such points in the studied chromosomal region whose nearest marker is within the k best scoring markers, where k is selected such that the resulting area has length at most t times the length of the studied region, and where k is maximal such value, or
those points in the studied chromosomal region whose nearest marker scores at least y or at most y, where y is scoring function dependent and is selected so that the probability of the gene being close to the marker is sufficiently large.

The location of the gene may also be determined by expert investigation of the marker scores or their visualization e.g. as a curve.

Permutation Tests

More information about the significance of the observed scores may be obtained by permutation tests. The results obtained by considering the marker frequencies or the linear model, as explained earlier, can be contrasted against the null hypothesis that all the persons are drawn from the same distribution; that is, there is no gene effect in the disease status. We propose to permute randomly the status fields of the persons, keeping the proportions of affected and control persons constant, in a fashion similar to the method of Churchill and Doerge (1994). We approximate marker-wise p values using permutations and then predict the DS gene to be in the vicinity of the marker with the smallest empirical p value. Consecutive markers are dependent, and thus a large number of mutually dependent p values are produced. This is not a problem, since we do not use the p values for hypothesis testing, but only for ranking markers.

Marker-wise p values are used to re-score markers by their statistical unexpectedness. The test is carried out as follows: The phenotypes of the persons are randomly shuffled a number (thousands) of times. The scores are re-calculated for each per-mutation mutation in turn. Marker-wise p value p(mᵢ) is the proportion of such permutation scores for marker mᵢ that are larger than or equal to the non-permuted score.

Each score s(mᵢ) is then refined by replacing it by the marker-wise p value p(mᵢ) of the score s(mᵢ).

Searching Several Genes

Several genes may be searched for simultaneously by using marker patterns that refer to several potential gene loci at the same time.

### EXAMPLES

Certain embodiments and results of the present invention are described in the following non-limiting examples.

### Example 1

### Simulated Data Sets

We evaluated the performance of the proposed HPM-G method with simulated data sets that correspond to a recently founded, relatively isolated founder subpopulation. Simulation of a population isolate was chosen, since it is recommended as the study population for LD studies. However, the method can be applied to any population that is suitable for LD analysis, since no assumptions are made about the population structure.

An isolated founder population, which grows from the initial size of 200 to 100,000 individuals in 20 generations, was simulated.

The population pedigree was first generated assuming distinct generations and ex-ponential growth of the population size. In each generation, the parents of the newborn individuals were randomly selected from members of the previous generation, with the exception that whenever a parent with at least one child was chosen, his/her spouse was always forced to become the other parent of the child. This procedure generates family structure into each generation.

In the simulation of inheritance, each member of the first generation was assigned to have one pair of homologous chromosomes. The genetic length of the chromosomes was 100 cM for both males and females. Meiosis was repeatedly simulated, and in each meiosis the number of crossover points was taken from a Poisson distribution with parameter value 1, which corresponds to the total genetic length of the chromosome. No chiasm interference was modeled. To accommodate the fact that ever-increasing informativeness of marker maps may soon facilitate whole-genome LD mapping, we used relatively dense and informative marker maps with inter-marker intervals of exactly one cM. Each marker contained 4 alleles, whose frequencies in the founder population were 0.4 for one allele and 0.2 for the remaining three alleles. The polymorphism information content (PIC) of each marker was thus fixed at 0.678.

To produce each of the 100 data sets for HPM-G and HPM, the processes of disease locus selection, diagnosing and sampling were done independently. Next, these processes are described.

For each data set, a random locus was selected as a disease locus, and 8 random chromosomes present in the original population were labeled as disease-carrying chromosomes. In the final population, all chromosomes that had inherited the disease locus identical-by-descent from one of the eight founders were considered to carry a disease-causing mutation.

In the diagnosing phase, we used a liability-based model, where an individual's probability of becoming affected depends on two factors: the presence of a disease mutation and a normally distributed random component. The random component is thought to contain factors such as environmental effects and effects of other, un-known genes. The liability of an individual is defined as L=5x₁+x₂+C, where indicator variable x₁ indicates the presence of any of the disease-causing mutations, and variable x₂ is randomly sampled from standard normal distribution. Based on the generated segment data, the value of constant C is set in such a way that the desired population prevalence of 5 per cent is reached. The liability value L defines the probability of an individual to be affected, denoted by p, via formula

$$log \frac{p}{1 - p} = L .$$

Having discovered the affection status of each individual, the desired number of individuals labeled as affected was randomly selected to constitute the affected sample.

The control samples were created using two different methods: for HPM-G that utilizes genotype data, the control individuals were simply taken randomly from the entire population. To do this, the sampling process described above was repeated, but this time the liability of each individual was purely random, including no genetic component.

For the original HPM that requires haplotype data, we used a more laborious sampling method: the genotypes of the parents of the affected individuals were collected to create family-based pseudocontrol chromosomes. This was done in practice by taking the alleles in the non-transmitted chromosomal segments of the parents of each affected individual and labeling them as control chromosomes. In reality, this is a common practice. In the simulations we treated the haplotypes obtained from the simulator as given, which corresponds to error-free haplotyping, and is expected to slightly favor HPM in the comparisons.

The simulation of missing data was based on the notion that in real genotyping laboratories there seems to be two types of clustering of missing data. First, missing genotypes tend to cluster to certain individuals, which can be a consequence of low quality samples. Second, certain markers may function poorly, likely producing missing genotypes. To mimic such clustering of missing data, we defined two parameters: parameter α corresponds to the amount of missing data that clusters to individuals and parameter β to the amount that clusters to markers. The missing genotypes were selected using the following procedure:

For each individual i, a personal missing genotype probability xᵢ¹ was computed as the x value of the first random point in (x, y) plane (x, yε[0,1]) that satisfies the inequality y<1/e^{αx}. Having computed the value of variable xᵢ¹ for the individual, each of his/her genotypes was then labeled as missing with probability xᵢ¹. In the second phase, the procedure was repeated for each marker. For each marker j, a marker failure probability xⱼ^{M} was computed in an analogous fashion as the x value of the first random point in (x, y) plane (x, yε[0,1]) that satisfies the inequality y<1/e^{βx} and each genotype corresponding to that marker was labeled as missing independently for each individual with probability xⱼ^{M}.

Values of variables α and β were empirically adjusted to produce the desired over-all levels of missing data. These values were: 25 and 80 for 5%, and 13 and 40 for 10% of missing data.

### Example 2

### Comparison to HPM

The localization accuracy was explored by plotting curves similar to power graphs:

the height of the curve shows the fraction of data sets for which the localization was successful, as a function of the length of the predicted region. The sample consisted of 150 affected and 150 control genotypes. The maximum length of a pattern was 7, and one gap of one marker was allowed. The association threshold was set to 10.

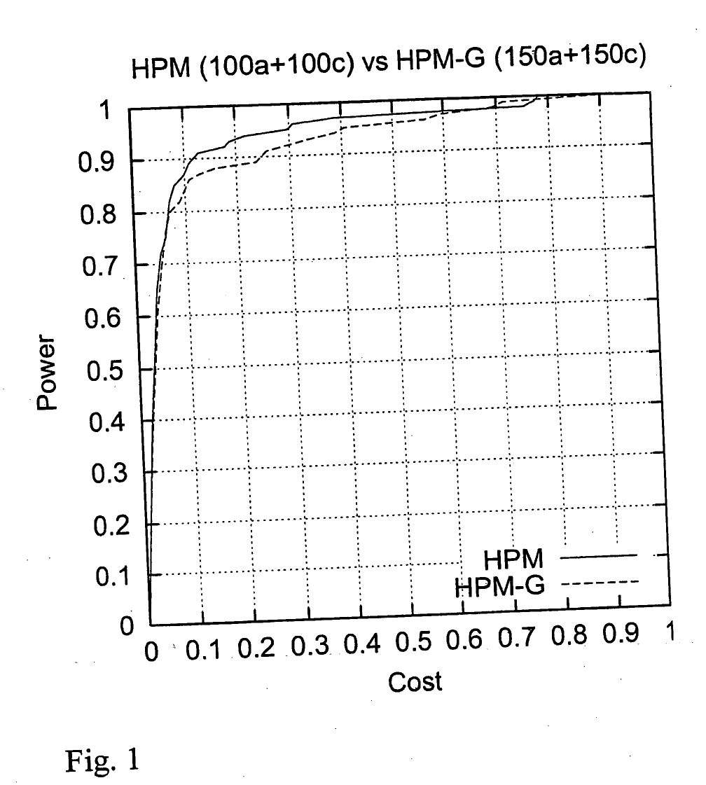

These numbers were based on experimentation. For comparison, we also show the corresponding curve for HPM with ⅓ smaller sample size, and thus equal genotyping cost (FIG. 1). With HPM we used association threshold 9, the parameters for the patterns were the same than those used with HPM-G.

The results show that HPM-G has a high accuracy, and that it is extremely competitive even in comparison to state-of-the-art methods that use explicitly haplotyped data.

### Example 3

### Effect of Sample Size

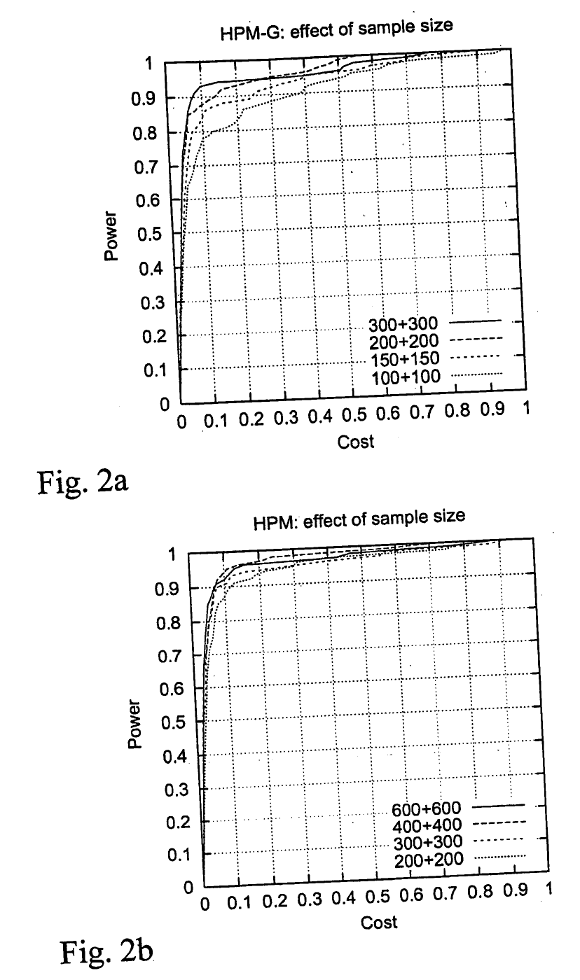

The effect of sample size was examined by experimenting with sample sizes of 100+100, 150+150, 200+200 and 300+300 people (FIG. 2a). FIG. 2b shows the corresponding results for HPM.

HPM-G performs well even with only 100+100 genotypes. On the other hand, if the amount of data is increased, the accuracy is improved.

### Example 4

### Effect of Missing Data

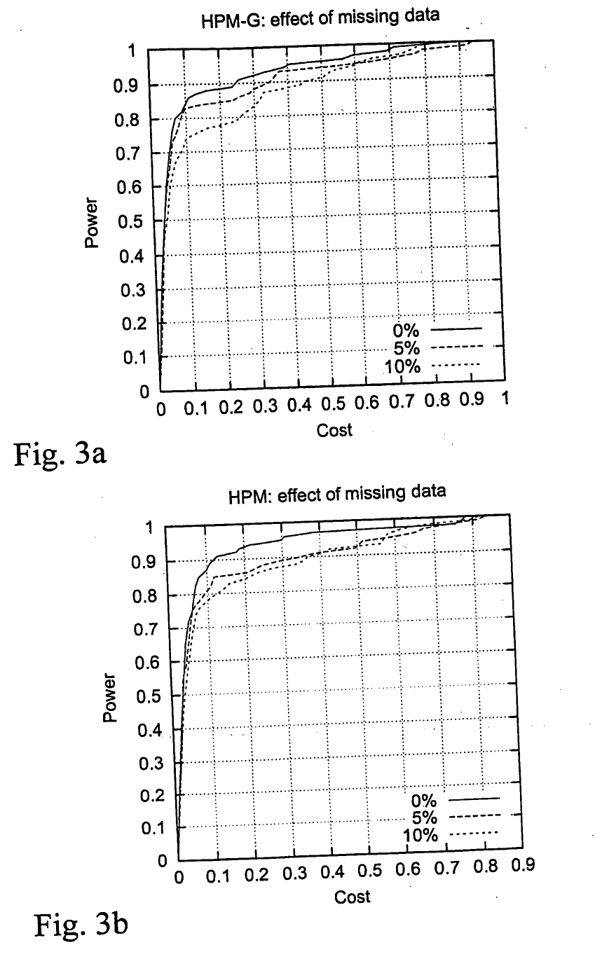

The influence of missing data was explored by randomly removing 5% or 10% of marker genotypes (FIG. 3a). FIG. 3b shows the corresponding results for HPM.

These results show that HPM-G is very robust against missing data.

### Example 5

### Localization Accuracy with Permutation Tests

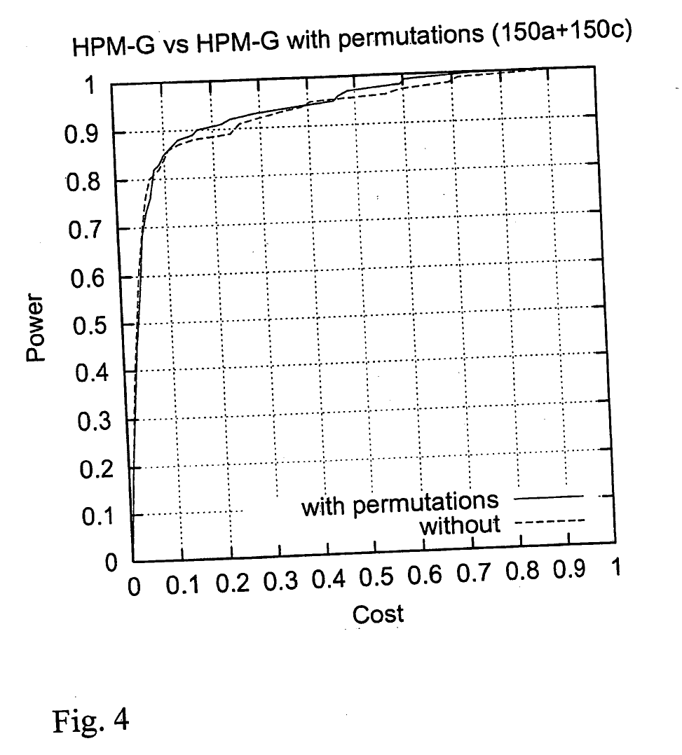

Permutation tests were used to obtain more information about the significance of observed marker frequencies. Marker-wise P values were used to sort markers by their statistical unexpectedness, not to test the statistical significance of the findings. We performed the following experiment in order to see if the prediction accuracy can be improved by permutation tests. We predicted the location of the DS gene to be at the marker with the smallest P value instead of the most frequent marker. The localization accuracy with 100 permutations compared to that without permutations is shown in FIG. 4. The curves are almost identical, which is due to the evenly distributed and identically informative markers.

The situation could be different with real marker data, where permutation tests are likely to bring a greater benefit.

### REFERENCES

Bain S, Todd J, Barnett A (1990) The British Diabetic Association—Warren repository. Autoimmunity 7:83-85
Churchill G A, Doerge R W (1994) Empirical threshold values for quantitative trait mapping. Genetics 138:963-971
Curtis D, North B V and Sham P C (2001) Use of an artificial neural network to detect association between a disease and multiple marker genotypes. Ann Hum Genet 65:95-107
Devlin B, Risch N, Roeder K (1996) Disequilibrium mapping: composite likelihood for pairwise disequilibrium. Genomics 36:1-16
Kruglyak L (1999) Prospects for whole-genome linkage disequilibrium mapping of common disease genes. Nat Genet 22:139-144
Kruglyak L, Daly M J, Reeve-Daly M P, Lander E S (1996) Parametric and nonparametric linkage analysis: a unified multipoint approach. Am J Hum Genet 58:1347-1363
Lazzeroni L C (1998) Linkage disequilibrium and gene mapping: an empirical least-squares approach. Am J Hum Genet 62:159-170
Mannila H, Toivonen H T T (1997) Levelwise search and borders of theories in knowledge discovery. Data Mining and Knowledge Discovery 1(3): 241-258
McPeek M S, Strahs A (1999) Assessment of linkage disequilibrium by the decay of haplotype sharing, with application to fine-scale genetic mapping. Am J Hum Genet 65:858-875
Service S K, Temple Lang D W, Freimer N B, Sandkuijl L A (1999) Linkage-disequilibrium mapping of disease genes by reconstruction of ancestral haplotypes in founder populations. Am J Hum Genet 64:1728-1738
Spielman, R S, McGinnish R E, Ewens, W J (1993) Transmission test for linkage dis-equilibrium: the insulin gene region and insulin-dependent diabetes mellitus (IDDM). Am J Hum Genet 52:506-515
Terwilliger J D (1995) A powerful likelihood method for the analysis of linkage dis-equilibrium between trait loci and one ore more polymorphic marker loci. Am J Hum Genet 56:777-787
Toivonen H T T, Onkamo P, Vasko K, Ollikainen V, Sevon P, Mannila H, Herr M and Kere J (2000) Data mining applied to linkage disequilibrium mapping. Am J Hum Genet 67:133-145
Toivonen H T T, Onkamo P, Vasko K, Ollikainen V, Sevon P, Mannila H, and Kere J (2000) Gene mapping by haplotype pattern mining. Proceedings of IEEE International Symposium on Bioinformatics and Biomedical Engineering, pp. 99-108, 10 October 2000
Zhang S, Zhang K, Li J and Zhao H (2002) On a family-based haplotype pattern mining method for linkage disequilibrium mapping. Web publication in Pacific Symposium on Biocomputing 2002, (http:/lwww .smi.stanford.edu/projects/helix/psb02/zhang.pdf)
Zhang and Zhao (2002) Linkage disequilibrium mapping with genotype data. Genetic Epidemiology 22:66-77

1-21. (canceled)

22. A method for gene mapping to locate a gene associated with certain phenotype from dataset of genotype and phenotype data, by analyzing associations between genetic markers mᵢ, which are polymorphic nucleic acid or protein sequences or strings of single-nucleotide polymorphisms deriving from a chromosomal region, comprising
i) searching from said dataset for all marker patterns P that satisfy a pattern evaluation function e(P), wherein
a. the marker patterns are expressions within said dataset comprising the marker-allele assignments and zero or more of the following: individual covariates, environmental variables and auxiliary phenotypes; and
b. the pattern evaluation function e(P) is true if and only if there is a strong association between the marker pattern P and the phenotype being studied,
by testing each marker of pattern P against the corresponding allele pair in genotype G, effectively finding out if there is a possible haplotype configuration of G which matches P and counting the possible matches as matches,
ii) scoring each marker mᵢ of said dataset with a marker score s(mᵢ), which is a function of the set Sᵢ defined as the set of marker patterns overlapping the marker mᵢ and satisfying the pattern evaluation function e as defined in step i), and
iii) locating said the gene to the marker mᵢ having the best score s(mᵢ) wherein the best score is the highest obtained score if said scoring function is designed to give higher scores closer to the gene, or the lowest obtained score if said scoring function is designed to give lower scores closer to the gene.

23. The method of claim 22, wherein a marker is scored as the sum of the weights of overlapping patterns.

24. The method of claim 23, wherein the weight of a pattern is a function of
the uncertainty of matching, e.g. 2^{1-N[i]}, where N[i] is the number of heterozygous markers within the pattern in genotype i, summed over all matched genotypes, or
the informativeness of the pattern, e.g. 2^{H}, where H is the average heterozygosity within the pattern, or
the strength of association, e.g. chi-squared.

25. The method of claim 22, wherein the marker patterns P are searched by the following algorithm:
Input
set U of possible marker patterns
evaluation function e(P) for patterns P in U
(generalization) relation<for patterns in U
where the function e and the relation<are such that if e(P) is true and P′<P, then e(P′) is also true
Output
set S={P ε U|e(P) is true} of patterns
Method
1. S : = { }
2. // Initialize the set of evaluated patterns:
3. E := { }
4. // Start with the most general patterns:
5. Gen := {P in U | there is no P′ in U, P′. □ P, such that P′ < P}
6. // Recursively evaluate patterns in a depth first order:
7. foreach P ∈ Gen { evaluatePatterns(P) }
8. end;
9. procedure evaluatePatterns(P) {
10. insert P into the set E
11. if e(P) = true then {
12. insert P into set S
13. // Find all specializations of P that have not been tested
14. // yet, and evaluate them recursively:
15. Spec := {P′ in U-E | P < P′, P′. □ P, and there is no P″ in
16. U-E, P″. □ P and P″. □ P′, with P < P″ < P′};
17. foreach P′ in Spec { evaluatePatterns(P′); }
18. }
19. }

26. The method of claim 22, wherein the marker patterns P are searched by the following algorithm:
Input
set U of possible marker patterns
evaluation function e(P) for patterns P in U
frequency threshold x
Output
set S={P in U| e(P) and ae(P) is true} of patterns, where ae(P) is true if and only if the frequency of pattern P exceeds a given threshold x
Method
20. S : = { }
21. // Initialize the set of evaluated patterns:
22. E := { }
23. // Start with the most general patterns:
24. Gen := {P in U | there is no P′ in U, P′ != P, such that P -> P′ }
25. // Recursively evaluate patterns in a depth-first order:
26. foreach P in Gen { evaluatePatterns(P) }
27. end
28. procedure evaluatePatterns(P) {
29. insert P into the set E
30. if ae(P) = true then {
31. if e(P) = true then insert P into set S
32. // Find all specializations of P that have not been tested
33. // yet, and evaluate them recursively:
34. Spec := {P′ in U-E | P′ -> P, P′. □ P, and there is no P″ in
35. U-E, P″. □ P and P″. □ P′, with P′ -> P″ and P″ -> P }
36. foreach P′ in Spec { evaluatePatterns(P′) }
37. }
38. }

27. The method of claim 22, wherein the marker patterns P are searched by the following algorithm:
Input
marker map M=(m₁, . . . , mₖ)
phenotype vector Y=(Y₁, . . . , Yₙ)
genotype matrix H of size n*k*2 (n persons, k markers, 2 alleles per person and marker)
association threshold x for chi-squared test
maximum pattern length l
maximum number of gaps g
maximum gap size s
Output
set S={P in U|e(P) is true} of patterns,
where U consists of patterns on M that consist of marker-allele assignments and that adhere to parameters l, g, and i, and
where e(P) is true if and only if chi-squared test on P using genotype matrix H and phenotypes Y exceeds the given threshold x
Method
39. S : = { }
40. // Number of case and control persons:
41. pi_{A} := number of affected persons;
42. pi_{C} := number of control persons;
43. pi := pi_{A} + pi_{C}
44. // A lower bound for pattern frequency:
45. lb := pi_{A} * pi * x / (pi_{C} * pi + pi_{A} * x)
46. // Variable for iterating over different patterns:
47. P = (p1, ... , pk) := (‘*’, ... , ‘*’)
48. for i := 1 to k {
49. // alleles(mᵢ) is the set of alleles of the i:th marker
50. foreach a in alleles(mᵢ) {
51. pᵢ := a
52. // Test pattern P and all its extensions:
53. checkPatterns(P, i, i, 0, 0)
54. // Reset pᵢ:
55. pᵢ := ‘*’
56. }
57. }
58. end
59. // Test haplotype pattern P and all patterns that can be generated
60. // by extending P from the right:
61. procedure checkPatterns(P, start, i, nr_of_gaps, gap_length) {
62. // Output strongly associated patterns
63. if chi-squared(P, M, H, Y) >= x and pᵢ != ‘*’ then insert P into set S
64. // Return if extended patterns would be too long:
65. if i = k or i+1-start > l then return
66. // Return if extended patterns can not be strongly disease-associated:
67. if frequency of P in affected persons is less than lb
68. then return;
69. // Create and test legal extensions of current pattern P (3 cases):
70. // 1. Give marker i+1 all possible values:
71. foreach a in alleles(mᵢ₊₁) {
72. pᵢ₊₁ := a
73. checkPatterns (P, start, i+1, nr_of_gaps, 0)
74. }
75. // 2. Introduce a new gap starting at marker i+1:
76. if pᵢ ≠ ‘*’ and nr_of_gaps < g and s ≧ 1 then {
77. pᵢ₊₁ := ‘*’
78. checkPatterns (P, start, i+1, nr_of_gaps+1, 1)
79. }
80. // 3. Extend the current gap over marker i+1:
81. if pᵢ = ‘*’ and gap_length < s then {
82. pᵢ₊₁ := ‘*’
83. checkPatterns (P, start, i+1, nr_of_gaps, gap_length+1)
84. }
85. // Before returning, reset pᵢ₊₁:
86. pᵢ₊₁ := ‘*’
87. return
88. }

28. The method of claim 22, wherein the marker patterns P are searched by the following algorithm:
Input
set U of possible marker patterns
evaluation function e(P) for patterns P in U
(generalization) relation<for patterns in U, where the function e and the relation<are such that if e(P) is true and P′<P, then e(P) is also true
Output
set S={P in U|e(P) is true} of patterns
Definitions
function Lgg: U→2^{U}, Lgg(P)={P′ in U|P>P′ and P′ !=P and there is no P″ in U such that P !=P″ !=P′ and P>P″>P′}, the set of least general generalizations of pattern P.
function Lss: U→2^{U}, Lss(P)={P′ in U|P<P′ and P′!=P and there is no P″ in U such that P !=P″!=P′ and P<P″<P′}, the set of least special specializations of pattern P.
Method
89. S : = { }
90. Q : = { }
91. // Start with the most general patterns:
92. F : = {P in U | there is no P′ in U, P′ != P, such that P′ < P};
93. while F != { } {
94. // Evaluate the candidate patterns:
95. foreach P in F {
96. if e(P) = true then insert P into set S
97. else remove P from set F
98. }
99. Q : = Q union F
100. // Generate a new set of candidate patterns:
101. C : = { }
102. foreach P in F {
103. C : = C union { P′ in U | P′ in Lss(P) and for all P″ in Lgg(P′):
104. P″ in Q }
105. }
106. F : = C
107. }
108. end

29. The method of claim 22, wherein the marker patterns P are searched by the following algorithm:
Input
set U of possible marker patterns
evaluation function e(P) for patterns P in U
frequency threshold x
Output
set S={P in U|e(P) and ae(P) is true} of patterns, where ae(P) is true if and only if the frequency of pattern P exceeds a given threshold x
Definitions
function Lgg: U→2^{U}, Lgg(P)={P′ in U|P→P′ and P′ !=P and there is no P″ in U such that P !=P″ !=P′ and P→P″−>P′}, the set of least general generalizations of pattern P.
function Lss: U→2^{U}, Lss(P)={P′ in U|P′→P and P′ !=P and there is no P″ in U such that P !=P″!=P′ and P′→P″→P}, the set of least special specializations of pattern P.
Method
109. S : = { }
110. Q : = { }
111. // Start with the most general patterns:
112. F := {P in U | there is no P′ in U, P′ != P, such that P -> P′ };
113. while F != { } {
114. // Evaluate the candidate patterns:
115. foreach P in F {
116. if ae(P) = true then {
117. if e(P) = true then insert P into set S
118. }
119. else remove P from set F
120. }
121. Q : = Q union F
122. // Generate a new set of candidate patterns:
123. C : = { }
124. foreach P in F {
125. C : = C union { P′ in U | P′ inLss(P) and for all P″ in Lgg(P′):
126. P″ in Q }
127. }
128. F : = C
129. }
130. end

30. The method of claim 22, wherein
a) the phenotype being studied is qualitative, and
b) the pattern evaluation function e(P) has the form e(P)=true ifand only if e′(P)>x, where e′(P) is the (signed) association measure χ² and x is a user-specified minimum value, which is chosen so that the sizes of Sᵢ are large enough, such as 7, to give statistically sufficiently reliable estimates for the gene locus, and
c) the score s(mᵢ) of marker mᵢ is the size of Sᵢ also called marker-wise pattern frequency of mᵢ and denoted by f(mᵢ).

31. The method of claim 22, wherein
a) the pattern evaluation function e(P) has the form e(P)=true if and only if e′(P)>x, where e′(P) is the absolute frequency of pattern P in the data and x is a user-specified value, which is chosen so that the sizes of Sᵢ are large enough, such as 20, to give statistically sufficiently reliable estimates for the gene locus, and,
b) in order to derive the score s(mᵢ), the p value (statistical significance) of each marker pattern P in determining the phenotype being studied is evaluated, and
c) the score s(mᵢ) is the distance between the observed p value distribution of patterns in Sᵢ and the uniform distribution, defined as average of (pᵢ−qᵢ)log (pᵢ/qᵢ) over all i=1 . . . n, where n is the number of haplotype patterns in Sᵢ,pᵢ is the ith smallest p value in Sᵢ, and qᵢ is the expectation of the ith smallest p value, if the p values were randomly drawn from the uniform distribution.

32. The method of claim 31, wherein the p value is computed using a linear model of
form Y=β₁X₁+ . . . +βₖXₖ+αZ+β₀, where the dependent variable Y is the phenotype being studied, X₁ through Xₖ are covariates, such as environmental factors, and Z is a dummy variable for the occurrence of the haplotype pattern, and
the coefficients α and β* are adjusted for best fit, and then the significance of Z as a covariate is assessed by using a t test with the null hypothesis “α=0”.

33. The method of claim 22, wherein each score s(mᵢ) is refmed by replacing it by the marker-wise p value of the score s(mᵢ), where the statistical significance of s(mᵢ) is measured against the null hypotheses that there is no gene effect.

34. The method of claim 33, wherein the marker-wise p values p(mᵢ) are determined by randomly permuting phenotypes.

35. The method of claim 22, wherein the area returned from the prediction of the gene location is contiguous or fragmented or a point.

36. The method of claim 22, wherein the location of the gene, predicted as a function of the scores s(mᵢ) and based on maximizing or minimizing the score, is predicted to the location of the marker mᵢ that maximizes or minimizes the marker score s(mᵢ).

37. The method of claim 22, wherein the location of the gene, predicted as a function of the scores s(mᵢ) and based on maximizing or minimizing the score, is predicted to the combination of most probable intervals for containing the trait-susceptibility locus that covers at most the desired proportion ranging from 0 to 100% of the region covered by markers mᵢ obtained by taking all such points in said region whose nearest marker is within the k best scoring markers, where k is selected such that the resulting area has length at most t times the length of said region, and where k is maximal such value.

38. The method of claim 22, wherein the location of the gene, predicted as a function of the scores s(mᵢ) and based on maximizing or minimizing the score, is predicted to those points in the studied chromosomal region whose nearest marker scores at leasty or at mosty, wherey is scoring function dependent and is selected so that the probability of the gene being close to the marker is sufficiently large.

39. The method of claim 22, wherein multiple genes are searched by using marker patterns that refers to different potential gene loci at the same time.

40. A computer-readable data storage medium having computer-executable program code stored operative to perform a method of claim 22 when executed on a computer.

41. A computer system, which is programmed to perform the method of claim 39.
